(19) United States
(12) Patent Application Publication
(10) Pub. No.: US 2011/0111459 A1
(43) Pub. Date: May 12, 2011

(54) COMPOSITIONS AND METHODS FOR NUCLEIC ACID DELIVERY

(75) Inventor: Frederick M. Boyce, Waltham, MA (US)

(73) Assignee: THE GENERAL HOSPITAL CORPORATION, Boston, MA (US)

(21) Appl. No.: 12/936,555

(22) PCT Filed: Apr. 20, 2009
(86) PCT No.: PCT/US2009/041086

§ 371 Date: Jan. 3, 2011

Related U.S. Application Data
(60) Provisional application No. 61/046,241, filed on Apr. 18, 2008.

(51) Int. Cl. C12P 21/02; C12N 5/071; C12N 15/85

(52) U.S. Cl. 435/69.1; 435/375; 435/458

(57) ABSTRACT

Disclosed herein are methods and compositions for preparing a lipid encapsulated nucleocapsid delivery composition capable of delivering a nucleic acid to a mammalian cell. The nucleocapsid delivery compositions disclosed herein are useful for large-scale protein production, expression of genes in a mammalian expression system, and/or pharmaceutical production of a recombinant protein for treatment of an individual. In addition, the nucleocapsid delivery compositions can be used to treat an individual to replace a gene or gene product, or alternatively to inhibit a gene product using an RNA interference molecule.

### CROSS REFERENCE TO RELATED APPLICATION

This application claims benefit under 35 U.S.C. §119(e) of the U.S. Provisional Application No. 61/046,241 filed Apr. 18, 2008, the contents of which are incorporated herein by reference in its entirety.

### FIELD OF THE INVENTION

The present invention relates to the field of exogenous gene delivery to a mammalian cell.

### BACKGROUND

Current methods for expressing an exogenous gene in a mammalian cell include the use of mammalian viral vectors, such as those derived from retroviruses, adenoviruses, herpes viruses, vaccinia viruses, polio viruses, or adeno-associated viruses. Other methods of expressing an exogenous gene in a mammalian cell include direct injection of DNA, the use of ligand-DNA conjugates, the use of adenovirus-ligand-DNA conjugates, calcium phosphate precipitation, and methods that utilize a liposome- or polycation-DNA complex. In some cases, the liposome- or polycation-DNA complex is able to target the exogenous gene to a specific type of tissue, such as liver tissue.

Typically, viruses that are used to express desired genes are constructed by removing unwanted characteristics from a virus that is known to infect, and replicate in, a mammalian cell. For example, the genes encoding viral structural proteins and proteins involved in viral replication are often removed to create a defective virus, and a therapeutic gene or gene of interest is then added. This principle has been used to create gene therapy vectors from many types of animal viruses such as retroviruses, adenoviruses, and herpes viruses. This method has also been applied to Sindbis virus, an RNA virus that normally infects mosquitoes but which can replicate in humans, causing a rash and an arthritis syndrome.

Non-mammalian viruses have been used to express exogenous genes in non-mammalian cells. For example, viruses of the family Baculoviridae (commonly referred to as baculoviruses) have been used to express exogenous genes in insect cells. One of the most studied baculoviruses is Autographa californica multiple nuclear polyhedrosis virus (AcMNPV). Although some species of baculoviruses that infect crustacea have been described (Blissard, et al., 1990, Ann. Rev. Entomology 35:127), the normal host range of the baculovirus AcMNPV is limited to the order lepidoptera. Baculoviruses have been reported to enter mammalian cells (Volkman and Goldsmith, 1983, Appl. and Environ. Microbiol. 45:1085-1093; Carbonell and Miller, 1987, Appl. and Environ. Microbiol. 53:1412-1417; Brusca et al., 1986, Intervirology 26:207-222; and Tjia et al., 1983, Virology 125:107-117). Although an early report of baculovirus-mediated gene expression in mammalian cells appeared, the authors later attributed the apparent reporter gene activity to the reporter gene product being carried into the cell after a prolonged incubation of the cell with the virus; a process known as “pseudotransduction” (Carbonell et al., 1985, J. Virol. 56:153-160; and Carbonell and Miller, 1987, Appl. and Environ. Microbiol. 53:1412-1417). These authors reported that, when the exogenous gene gains access to the cell as part of the baculovirus genome, the exogenous gene is not expressed de novo. Subsequent studies have demonstrated de novo baculovirus-mediated gene expression in mammalian cells (Boyce and Bucher, 1996, Proc. Natl. Acad. Sci. 93:2348-2352). In addition to the Baculoviridae family, there exist other families of viruses in nature that multiply mainly in invertebrates. These viruses can be used in a manner similar to baculoviruses as described above.

Gene therapy methods are currently being investigated for their usefulness in treating a variety of disorders. Most gene therapy methods involve supplying an exogenous gene to overcome a deficiency in the expression of a gene in the patient. Other gene therapy methods are designed to counteract the effects of a disease. Still other gene therapy methods involve supplying a nucleic acid (e.g., antisense RNA or an RNA interference molecule) to inhibit expression of a gene of the host cell (e.g., an oncogene) or expression of a gene from a pathogen (e.g., a virus).

Certain gene therapy methods are being examined for their ability to correct inborn errors of metabolism, e.g., errors of the urea cycle (see, e.g., Wilson et al., 1992, J. Biol. Chem. 267: 11483-11489). The urea cycle is the predominant metabolic pathway by which nitrogen wastes are eliminated from the body. The steps of the urea cycle are primarily limited to the liver, with the first two steps occurring within hepatic mitochondria. In the first step, carbamoyl phosphate is synthesized in a reaction which is catalyzed by carbamoyl phosphate synthetase I (CPS-I). In the second step, citrulline in formed in a reaction catalyzed by ornithine transcarbamylase (OTC). Citrulline then is transported to the cytoplasm and condensed with aspartate into arginosuccinate by arginosuccinate synthetase (AS). In the next step, arginosuccinate lyase (ASL) cleaves arginosuccinate to produce arginine and fumarate. In the last step of the cycle, arginase converts arginine into ornithine and urea.

A deficiency in any of the five enzymes involved in the urea cycle has significant pathological effects, such as lethargy, poor feeding, mental retardation, coma, or death within the neonatal period (see, e.g., Emery et al., 1990, In: Principles and Practice of Medical Genetics, Churchill Livingstone, New York). OTC deficiency usually manifests as a lethal hyperammonemic coma within the neonatal period. A deficiency in AS results in citrullinemia which is characterized by high levels of citrulline in the blood. The absence of ASL results in arginosuccinic aciduria (ASA), which results in a variety of conditions including severe neonatal hyperammonemia and mild mental retardation. An absence of arginase results in hyperarginemia which can manifest as progressive spasticity and mental retardation during early childhood. Other current used therapies for hepatic disorders include dietary restrictions; liver transplantation; and administration of arginine freebase, sodium benzoate, and/or sodium phenylacetate.

### SUMMARY OF THE INVENTION

The lipid encapsulated nucleocapsid delivery compositions described herein can be used to transduce mammalian cells or cell lines in order to express proteins for large-scale production, for gene therapy and as a research tool (e.g., for high throughput screening). In one embodiment of the methods and compositions described herein, the lipid encapsulated nucleocapsid is produced from a baculoviral vector. Nucleocapsid delivery compositions described herein are particularly useful for transducing cells or cell lines that remain relatively refractory to infection with unmodified non-mammalian vectors. The methods disclosed herein provide a means for transducing a mammalian cell with an exogenous nucleic acid sequence, including a mammalian cell which is otherwise refractory to existing vectors.

The compositions and methods described herein are useful for expressing an exogenous gene(s) in a mammalian cell (e.g., a cultured hepatocyte such as HepG2). This method can be employed in the manufacture of proteins, such as proteins which are used pharmaceutically. The method can also be used therapeutically. For example, the compositions and methods disclosed herein can be used to express in a patient a gene encoding a protein which corrects a deficiency in gene expression.

One aspect of the methods disclosed herein relates to a method for preparing a nucleic acid delivery composition, the method comprising contacting a purified nucleocapsid with a lipid, wherein the purified nucleocapsid is isolated from a non-mammalian virus and wherein the outer envelope of the non-mammalian virus has been removed prior to said contacting, whereby a lipid encapsulated nucleocapsid delivery composition capable of delivering a nucleic acid to a mammalian cell is produced.

Another aspect of the methods disclosed herein involves preparing a lipid encapsulated nucleocapsid delivery composition by removing the outer envelope of an isolated non-mammalian virus to produce a purified nucleocapsid, and contacting the purified nucleocapsid with a lipid, such that a lipid encapsulated nucleocapsid delivery composition is produced.

In one embodiment of this aspect and all other aspects described herein, the lipid encapsulated nucleocapsid delivery composition is produced from an insect virus. In another embodiment of this aspect and all other aspects described herein, the lipid encapsulated nucleocapsid delivery composition is produced from a baculovirus.

In another embodiment of this aspect and all other aspects described herein, the lipid encapsulated nucleocapsid delivery composition comprises a nucleic acid that encodes a heterologous gene.

In another embodiment of this aspect and all other aspects described herein, the step of removing the outer envelope comprises contacting the isolated non-mammalian virus with an organic solvent.

In another embodiment of this aspect and all other aspects described herein, the step of contacting the purified nucleocapsid with a lipid further comprises sonicating the purified nucleocapsid in the presence of the lipid.

Another aspect disclosed herein is a method for producing a recombinant protein of interest, comprising contacting a mammalian cell with a lipid encapsulated nucleocapsid delivery composition, wherein the nucleocapsid delivery composition comprises a nucleic acid encoding a recombinant protein of interest, wherein contacting results in transduction of the cell with the lipid encapsulated nucleocapsid delivery composition.

In one embodiment of this aspect and all other aspects described herein, the method further comprises isolating the recombinant protein from the mammalian cell.

In another embodiment of this aspect and all other aspects described herein, the recombinant protein is a therapeutic protein.

In another embodiment of this aspect and all other aspects disclosed herein, the mammalian cell is comprised by a mammal. In another embodiment of this aspect and all other aspects described herein, the mammalian cell is not comprised by a mammal.

Another aspect disclosed herein is a method for introducing a nucleic acid to a mammalian cell, the method comprising contacting a mammalian cell with a lipid encapsulated nucleocapsid delivery composition, the delivery composition further comprising a nucleic acid encoding a recombinant protein of interest, wherein contacting results in introduction of the nucleic acid to the mammalian cell.

Another aspect disclosed herein is a method for introducing a nucleic acid to a mammalian cell, which comprises contacting a mammalian cell with a lipid encapsulated nucleocapsid delivery composition, wherein the nucleocapsid delivery composition is modified by the steps of: (a) removing the outer envelope of the isolated non-mammalian virus to produce a purified nucleocapsid, and contacting the purified nucleocapsid with a lipid to produce a lipid encapsulated nucleocapsid composition.

In one embodiment of this aspect and all other aspects disclosed herein, the lipid encapsulated nucleocapsid delivery composition comprises a nucleic acid that encodes a protein of interest. In a preferred embodiment the protein of interest comprises a therapeutic protein.

Also disclosed herein is a lipid encapsulated nucleocapsid delivery composition, the composition produced by the steps of: (a) removing the outer envelope of an isolated non-mammalian virus to produce a purified nucleocapsid, and contacting the purified nucleocapsid with a lipid to produce a lipid encapsulated nucleocapsid delivery composition capable of delivering a nucleic acid to a mammalian cell.

Also disclosed herein is a lipid encapsulated nucleocapsid delivery composition, which comprises a purified nucleocapsid isolated from a non-mammalian virus, and an exogenous lipid composition.

In one embodiment, the lipid is physically associated with the purified nucleocapsid. In another embodiment, the lipid composition substantially encapsulates the purified nucleocapsid.

In a preferred embodiment the nucleocapsid further comprises a nucleic acid that encodes a protein of interest, e.g., a recombinant protein of interest.

In some embodiments, the lipid encapsulated nucleocapsid delivery composition further comprises a targeting moiety, e.g., a protein that directs the nucleocapsid delivery composition to a desired mammalian target cell.

Also described herein is a liposome encapsulated nucleocapsid composition comprising a purified nucleocapsid isolated from a non-mammalian virus encapsulated in a liposome.

In another aspect, the methods described herein relate to a method for preparing a liposome encapsulated nucleocapsid delivery composition, the method comprising: (a) removing the outer envelope of an isolated non-mammalian virus to produce a purified nucleocapsid; (b) contacting said nucleocapsid with a lipid to form a mixture; and (c) treating said mixture to induce liposome formation, wherein said nucleocapsid is encapsulated by said liposome and wherein said liposome encapsulated nucleocapsid is capable of delivering a nucleic acid to a mammalian cell.

### Definitions

As used herein, the term “nucleocapsid” refers to the central core of an enveloped non-mammalian virus that remains after substantial removal of the envelope components. A nucleocapsid necessarily comprises the nucleic acid component of the virus and core protein or proteins that package the nucleic acid in a particle. In specific embodiments, one or more components of the nucleocapsid can be modified (e.g., genetically modified).

As used herein, the term ‘removing the outer envelope’ relates to the modification of a non-mammalian virus (comprising a nucleocapsid enveloped by a lipid membrane) wherein the viral coat (also denoted herein as ‘outer envelope’, ‘viral coat’ and ‘viral envelope’) is removed to produce a ‘purified’ nucleocapsid. Removing the outer envelope can be performed, for example by extraction of the nucleocapsid using an organic solvent. The resulting ‘purified nucleocapsid’ is further modified using the methods described herein. In reference to a ‘purified nucleocapsid’, it is preferred that at least 75% of the lipids comprised by the outer envelope are removed from the nucleocapsid, preferably at least 80%, at least 85%, at least 90%, at least 95%, or at least 99% of the lipids comprised by the outer envelope are removed from the nucleocapsid. Ideally, the term ‘purified’ refers to the substantially complete removal of the lipids comprised by the outer envelope. Thus, a “lipid encapsulated nucleocapsid” is a viral nucleocapsid that is 1) partially or completely devoid of endogenous viral envelope, and 2) is associated with an exogenous lipid composition. A lipid encapsulated nucleocapsid can be produced using the nucleocapsid from an occluded virus or a budded virus.

The term ‘exogenous lipid’ is used to describe a lipid composition that is substantially free of pre-existing cellular protein from the native virus or virus to be modified (e.g., baculovirus coat proteins), and permits the formation of a lipid layer either spontaneously or by external stimulation (e.g., sonication). It is preferred that the lipid is not derived from the non-mammalian virus to be modified. In specific embodiments, the lipid composition may comprise other non-lipid components, which facilitate or enhance the transduction process. In other specific embodiments, the lipid composition consists entirely of lipids, that is, the lipid layer does not comprise a protein component. The lipid can be present in a single layer, a bilayer, or in a more complex arrangement, e.g., a plurality of concentric or non-concentric layers. In preferred embodiments, the lipid can be a transfection reagent obtained from a commercial source. In some embodiments, the lipid composition comprises a mixture of more than one lipid. Upon association of the nucleocapsid and the exogenous lipid composition, the lipid is also referred to herein as an “artificial coat”, “artificial viral coat”, or an “artificial outer membrane”. A purified nucleocapsid with an artificial coat is referred to herein as a “lipid encapsulated nucleocapsid” or a “nucleocapsid delivery composition.” The term “nucleocapsid delivery composition” is used interchangeably herein with the term “nucleic acid delivery composition.”

As used herein, the term “transduce” means that a nucleic acid delivery composition introduces its nucleic acid to a cell in a manner that results in expression of a gene encoded by the introduced nucleic acid. In one embodiment, a purified non-mammalian nucleocapsid can be used to produce a delivery package having a modified host range and/or modified tropism relative to the virus from which the nucleocapsid was isolated.

Thus, as used herein, the term “capable of delivering nucleic acid to a mammalian cell” refers to the ability to transduce a mammalian cell as the term “transduce” is used herein.

By “heterologous” gene or promoter is meant any gene or promoter that is not normally part of the non-mammalian viral genome. Such genes or promoters/regulatory elements can include those genes that are normally present in the mammalian cell to be transduced; also included are genes or regulatory elements that are not normally present in the mammalian cell to be transduced (e.g., related and unrelated genes of other cells or species). The term ‘exogenous’ in reference to a gene is also used herein in the same manner as the term ‘heterologous’.

By “promoter” is meant at least a minimal sequence sufficient to direct transcription. A “mammalian-active” promoter is one that is capable of directing transcription in a mammalian cell. The term “mammalian-active” promoter includes promoters that are derived from the genome of a mammal, i.e., “mammalian promoters,” and promoters of viruses that are capable of directing transcription in mammals (e.g., an MMTV promoter, RSV promoter etc.). Other promoters that are useful in the methods and compositions described herein include those promoters that are sufficient to render promoter-dependent gene expression controllable for cell-type specificity, cell-stage specificity, or tissue-specificity (e.g., liver-specific promoters), and those promoters that are “inducible” by external signals or agents (e.g., metallothionein, MMTV, and pENK promoters); such elements can be located in the 5′ or 3′ regions of the native gene. The promoter sequence can be one that does not occur in nature, so long as it functions in a mammalian cell. An “inducible” promoter is a promoter that, (a) in the absence of an inducer or inducing conditions, does not direct expression, or directs low levels of expression, of a gene to which the inducible promoter is operably linked; or (b) prohibits expression in the presence of a regulating factor (i.e., repressor) that, when removed, permits increased expression from the promoter (e.g., the tet system).

The term ‘physically associated’ is used herein to describe the relationship of exogenous lipid to a nucleocapsid isolated from a non-mammalian virus. The lipid can be in direct contact with the nucleocapsid such that they can physically touch. It is preferred that the exogenous lipid substantially encapsulates the nucleocapsid.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth.

### DETAILED DESCRIPTION

The methods described herein are useful for preparing a lipid encapsulated nucleocapsid and optionally using such a composition to deliver an exogenous gene(s) to a mammalian cell in vitro or in vivo. This method can be employed in the manufacture of proteins to be purified, such as proteins that are administered as pharmaceutical agents (e.g., insulin). The compositions described herein can also be used therapeutically. For example, the nucleocapsid delivery compositions described herein can be administered to an individual or to a cell to permit expression of a gene to correct a deficiency in gene expression. In alternative methods of therapy, the compositions and methods described herein can be used to express any protein, RNA interference molecule (e.g., antisense RNA, miRNA, siRNA, shRNA among others), or peptide fragment in a cell. Methods for use of RNA interference molecules are known to those of skill in the art and can be found in, for example Coburn, G. and Cullen, B. (2002) J. of Virology 76(18):9225; Stewart, et al. (2003) RNA Apr; 9(4):493-501; Jackson et al. Nature Biotechnology 6:635-637, 2003; Braasch et al., Biochemistry, 42: 7967-7975, 2003; Xia, H. et al. (2002) Nat Biotechnol 20(10):1006; and Rubinson, D. A., et al. ((2003) Nat. Genet. 33:401-406.

### Viruses

Advantages

The lipid encapsulated nucleocapsids described herein have an enhanced ability to transduce and express a gene in a mammalian cell compared to a non-mammalian virus, which is due in part to the use of exogenous lipids comprising an artificial outer envelope of the purified nucleocapsid. Thus, the lipid encapsulated nucleocapsids described herein are capable of delivering a nucleic acid to a broader range of host species (e.g., human, mammals, insects, etc,) and/or a broader range of target cell types (e.g., cardiomyocyte, skeletal myocyte, fibroblast, hepatic cell etc.) than the host range and/or cell type range of an unmodified non-mammalian virus. If desired, the lipid encapsulated nucleocapsid can be prepared such that the nucleocapsid delivery composition has a high specificity for a particular cell type, permitting targeted delivery to a desired tissue. The lipid encapsulated nucleocapsids described herein have a greater capability for transducing a desired cell relative to a non-mammalian virus. The methods and compositions described herein allow for de novo expression of an exogenous gene (e.g., β-galactosidase) in a transduced cell and permits protein to be actively synthesized in the cell, as opposed to being carried along with the nucleocapsid passively.

The non-mammalian viruses used in the preparation of lipid encapsulated nucleocapsid compositions described herein are not normally pathogenic to humans; thus, concerns about safe handling of these viruses are minimized. Similarly, because the majority of naturally-occurring viral promoters are not normally active in a mammalian cell, production of undesired viral proteins is inhibited. For example, PCR-based experiments indicate that some viral late genes are not expressed. Accordingly, in contrast to some mammalian virus-based gene therapy methods, the lipid encapsulated nucleocapsids described herein should not provoke a host immune response to the viral proteins. In addition, lipid encapsulated nucleocapsid delivery compositions can be propagated with cells grown in serum-free media, eliminating the risk of adventitious infectious agents present in the serum contaminating the preparation. In addition, the use of serum-free media eliminates a significant expense faced by users of mammalian viruses. Certain non-mammalian viruses, such as baculoviruses, can be grown to a high titer (i.e., 10⁸ pfu/ml). Generally, viral genomes are large (e.g., the baculovirus genome is 130 kbp); thus, viruses used in the methods and compositions described herein can accommodate large exogenous DNA molecules. In certain embodiments, the methods and compositions described herein employ a virus whose genome has been engineered to contain an exogenous origin of replication (e.g., the EBV oriP). The presence of such sequences on the virus genome can allow episomal replication of the viral nucleic acid, increasing persistence in the transduced cell. Where the lipid encapsulated nucleocapsid described herein is used in the manufacture of proteins to be purified from the cell, the methods and compositions described herein offer the advantage that it employs a mammalian expression system. Accordingly, one can expect proper post-translational processing and modification (e.g., glycosylation) of the gene product.

Exemplary Viruses

Exemplary viruses for use in producing a lipid encapsulated nucleocapsid using the methods described herein include the Baculoviridae family of viruses, especially Autographa californica.

Viruses other than the baculovirus Autographa californica can be used in the methods disclosed herein. For example, Bombyx mori nuclear polyhedrosis virus, Orgyia pseudotsugata mononuclear polyhedrosis virus, Trichoplusia ni mononuclear polyhedrosis virus, Helioththis zea baculovirus, Lymantria dispar baculovirus, Cryptophlebia leucotreta granulosis virus, Penaeus monodon-type baculovirus, Plodia interpunctella granulosis virus, Mamestra brassicae nuclear polyhedrosis virus, and Buzura suppressaria nuclear polyhedrosis virus can be used.

Nuclear polyhedrosis viruses, such as multiple nucleocapsid viruses (MNPV) or single nucleocapsid viruses (SNPV), are preferred. In particular, Choristoneura fumiferana MNPV, Mamestra brassicae MNPV, Buzura suppressaria nuclear polyhedrosis virus, Orgyia pseudotsugata MNPV, Bombyx mori SNPV, Heliothis zea SNPV, and Trichoplusia ni SNPV can be used.

Granulosis viruses (GV), such as the following viruses, are also included among those that can be used in the methods and compositions described herein: Pieris brassicae GV, Artogeia rapae GV, and Cydia pomonella granulosis virus (CpGV). Also, non-occluded baculoviruses (NOB), such as Heliothis zea NOB and Oryctes rhinoceros virus can be used.

Other insect (e.g., lepidopteran) and crustacean viruses can also be used in the methods and compositions described herein. Further examples of useful viruses include those that infect fungi (e.g., Strongwellsea magna) and spiders. Viruses that are similar to baculoviruses have been isolated from mites, Crustacea (e.g., Carcinus maenas, Callinectes sapidus, the Yellow Head Baculovirus of penaeid shrimp, and Penaeus monodon-type baculovirus), and Coleoptera. Also useful in the methods and compositions described herein is the Lymantria dispar baculovirus.

If desired, the baculovirus genome can be engineered to carry a human origin of replication; such sequences have been identified (Burhans et al., 1994, Science 263: 639-640) and can facilitate replication in human cells. Other origins of replication, such as the Epstein-Barr Virus replication origin and trans-acting factor, can facilitate gene expression in human cells. Optionally, the baculovirus can be engineered to express more than one exogenous gene. If desired, the lipid encapsulated nucleocapsid can be engineered to facilitate targeting of the nucleocapsid delivery composition to certain cell types. For example, ligands which bind to cell surface receptors can be expressed or placed on the surface of the lipid encapsulated nucleocapsid composition. Alternatively, the lipid encapsulated nucleocapsid can be chemically modified to target the nucleocapsid delivery composition to a particular receptor.

Certain non-mammalian viruses (e.g., baculoviruses) may be occluded in a protein inclusion body, or they may exist in a plasma membrane budded form. Where an occluded virus is used in the methods and compositions described herein, the virus may first be liberated from the protein inclusion body, if desired. Conventional methods employing alkali may be used to release the virus (O'Reilly et al., 1992, In: Baculovirus expression vectors, W. H. Freeman, New York). An occluded, alkali-liberated baculovirus may be taken up by a cell more readily than is the non-occluded budded virus (Volkman and Goldsmith, 1983, Appl. and Environ. Microbiol. 45:1085-1093).

A non-mammalian viral (e.g., baculoviral) genome, which is capable of integrating into a chromosome of the host cell may also be used in the methods and compositions described herein. Such an integrating viral genome may persist in the cell longer than that of a non-integrating virus. Accordingly, methods of gene expression involving such viral genomes may obviate the need for repeated administration of the lipid encapsulated nucleocapsid to the cell, thereby decreasing the likelihood of mounting an immune response to the delivery composition. In other embodiments, it can be advantageous to use a lipid encapsulated nucleocapsid which cannot integrate its genetic material into the host genome.

Conventional methods can be used to propagate the viruses used in the methods and compositions described herein (see, e.g., Burleson, et al., 1992, Virology: A Laboratory Manual, Academic Press, Inc., San Diego, Calif. and Mahy, ed., 1985, Virology: A Practical Approach, IRL Press, Oxford, UK). For example, a baculovirus can be plaque purified and amplified according to standard procedures (see, e.g., O'Reilly et al. infra and Summers and Smith, 1987, A manual of methods for baculovirus vectors and insect cell culture procedures, Texas Agricultural Experiment Station Bulletin No. 1555, College Station, Tex.). Amplified virus can be concentrated by ultracentrifugation in an SW28 rotor (24,000 rpm, 75 minutes) with a 27% (w/v) sucrose cushion in 5 mM NaCl, 10 mM Tris pH 7.5, and 10 mM EDTA. The viral pellet can then be re-suspended in phosphate-buffered saline (PBS) and sterilized by passage through a 0.45 μ.m filter (Nalgene). If desired, the virus can be re-suspended by sonication in a cup sonicator. Viral titers for non-mammalian viruses used as starting material for lipid encapsulated nucleocapsid delivery compositions can be assayed by plaque assay on appropriate host cells.

Genetic manipulation of a baculovirus for use in the methods and compositions described herein can be accomplished with commonly-known recombination techniques originally developed for expressing proteins in baculovirus (see, e.g., O'Reilly et al., 1992, In: Baculovirus expression vectors, W. H. Freeman, New York), and is described in further detail herein.

### Preparing a Lipid Encapsulated Nucleocapsid

Provided herein are methods for preparing a nucleocapsid delivery composition such that it has an increased tropism and can transduce mammalian cells. The following describes steps and considerations for this process.

Removal of the outer lipid envelope of the viruses described herein can be achieved by any number of ways, including for example an organic solvent extraction of the lipid. For such a method the isolated virus is first mixed thoroughly with an organic solvent (e.g., ethyl ether, chloroform), for example by vortexing the mixture. The admixture is then centrifuged such that the organic and aqueous phases separate from one another to form distinct layers. The nucleocapsids are isolated by mechanically removing the layer at the interphase of the organic and aqueous layers. The organic phase, with the hydrophobic lipids, is discarded. The isolated and purified nucleocapsids are re-suspended in a saline solution, such as phosphate buffered saline, and subsequently re-coated with an exogenous lipid composition.

The exogenous lipid composition is then added to the purified nucleocapsids to form an admixture in order to produce a lipid encapsulated nucleocapsid composition. In some cases it is beneficial to stimulate the physical association of the lipids with the nucleocapsids by an external stimulus, such as sonication. It is preferred that the exogenous lipid composition used herein, is not obtained directly from the step of removing the outer envelope of the virus.

While it is not absolutely necessary, in preferred embodiments, the exogenous lipid compositions are obtained from a commercial source. Such preferred lipids include, for example Lipofectamine™ (Invitrogen; Carlsbad, Calif.), Lipofectamine 2000™ (Invitrogen; Carlsbad, Calif.), 293fectin™ (Invitrogen; Carlsbad, Calif.), Cellfectin™ (Invitrogen; Carlsbad, Calif.), DMRIE-C™ (Invitrogen; Carlsbad, Calif.), FreeStyle™ MAX (Invitrogen; Carlsbad, Calif.), Lipofectamine™ 2000 CD (Invitrogen; Carlsbad, Calif.), Lipofectamine™ (Invitrogen; Carlsbad, Calif.), RNAiMAX (Invitrogen; Carlsbad, Calif.), Oligofectamine™ (Invitrogen; Carlsbad, Calif.), Optifect™ (Invitrogen; Carlsbad, Calif.), X-tremeGENE Q2 Transfection Reagent (Roche; Grenzacherstrasse, Switzerland), DOTAP Liposomal Transfection Reagent (Grenzacherstrasse, Switzerland), DOSPER Liposomal Transfection Reagent (Grenzacherstras se, Switzerland), or Fugene (Grenzacherstras se, Switzerland), Transfectam® Reagent (Promega; Madison, Wis.), TransFast™ Transfection Reagent (Promega; Madison, Wis.), Tfx™-20 Reagent (Promega; Madison, Wis.), Tfx™-50 Reagent (Promega; Madison, Wis.), DreamFect™ (OZ Biosciences; Marseille, France), EcoTransfect (OZ Biosciences; Marseille, France), TransPassa D1 Transfection Reagent (New England Biolabs; Ipswich, Mass., USA), LyoVec™/LipoGen™ (Invivogen; San Diego, Calif., USA), PerFectin Transfection Reagent (Genlantis; San Diego, Calif., USA), NeuroPORTER Transfection Reagent (Genlantis; San Diego, Calif., USA), GenePORTER Transfection reagent (Genlantis; San Diego, Calif., USA), GenePORTER 2 Transfection reagent (Genlantis; San Diego, Calif., USA), Cytofectin Transfection Reagent (Genlantis; San Diego, Calif., USA), BaculoPORTER Transfection Reagent (Genlantis; San Diego, Calif., USA), TrojanPORTER™ transfection Reagent (Genlantis; San Diego, Calif., USA), RiboFect (Bioline; Taunton, Mass., USA), PlasFect (Bioline; Taunton, Mass., USA), UniFECTOR (B-Bridge International; Mountain View, Calif., USA), SureFECTOR (B-Bridge International; Mountain View, Calif., USA), or HiFect™ (B-Bridge International, Mountain View, Calif., USA), among others.

Some non-limiting examples of lipids (and derivatives thereof) useful with the methods and compositions described herein include LN-[1-(2,3-Dioleyloxy)propyl]-N,N,N-trimethylammonium chloride (see e.g., U.S. Pat. No. 5,676,954, which is herein incorporated by reference in its entirety), N-(2,3-di-(9-(Z)-octadecenyloxy))-prop-1-yl-N,N,N-trimethylammonium chloride (DOTMA); N-(2,3-di-octadecyloxy)-prop-1-yl-N,N,N-triemethylammonium chloride; N-(2,3-di-(4-(Z)-decenyloxy))-prop-1-yl-N,N,N-trimethylammonium chloride; N-(2,3-di-hexadecyloxy)-prop-1-yl-N,N,N-trimethylammonium chloride (“BISHOP”); N-(2,3-di-decyloxy)-prop-1-yl-N,N,N-trimethylammonium chloride; N-(2-hexadecyloxy-3-decyloxy)-prop-1-yl-N,N,N-trimethylammonium chloride; N-(2-hexadecyloxy-3-decyloxy)-prop-1-yl-N,N-dimethylamine hydrochloride; N-(9,10-di-decyloxy)-dec-1-yl-N,N,N-trimethylammonium chloride; N-(5,6-di-(9-(Z)-octadecenyloxy))-hex-1-yl-N,N,N-trimethylammonium chloride; N-(3,4-di-(9-(Z)-octadecenyloxy))-but-1-yl-N,N,N-trimethylammonium chloride (see e.g., U.S. Pat. No. 4,897,355, which is incorporated herein in its entirety); N-(ω,(ω-1)-dialkyloxy-N,N,N-tetrasubstituted ammonium (see e.g., U.S. Pat. Nos. 4,946,787, 5,049,386), N-(ω,(ω-1)-dialkenyloxy-N,N,N-tetrasubstituted ammonium (see e.g., U.S. Pat. Nos. 4,946,787, 5,049,386, which are herein incorporated by reference in their entirety); 1,2-O-dioleyl-3-dimethylamino propyl-β-hydroxyethylammonium acetate (DORI diether) (see e.g., U.S. Pat. No. 5,459,127, which is herein incorporated by reference in its entirety); DL 1,2-O-dipalmityl-3-dimethylaminopropyl-β-hydroxyethylammonium acetate (DPRI diether) (see e.g., U.S. Pat. No. 5,459,127); DL-1,2-dioleoyl-β-dimethylaminopropyl-β-hydroxyethylammonium acetate (DORI diester) (see e.g., U.S. Pat. No. 5,459,127); DL-1,2-dipalmitoyl-3-dimethylaminopropyl-β-hydroxyethylammonium (DPRI diester) (see e.g., U.S. Pat. No. 5,459,127); DL-1,2-dioleoyl-3-propyltrimethylammonium chloride (DOTAP) (see e.g., U.S. Pat. No. 5,459,127); 1,2-dipalmitoyl-3-propyltrimethylammonium chloride (DPTMA diester) (see e.g., U.S. Pat. No. 5,459,127); 3,5-(N,N-di-lysyl)-diaminobenzoyl-3-(DL-1,2-dipalmitoyl-dimethylaminopropyl-β-hydroxyethylamine) (DLYS-DABA-DPRI diester) (see e.g., U.S. Pat. No. 5,459,127); 3,5-(N,N-di-lysyl)-diaminobenzoyl-glycyl-3-(DL-1,2-dipalmitoyl-dimethylaminopropyl-β-hydroxyethylamine) (DLYS-DABA-GLY-DPRI diester) (see e.g., U.S. Pat. No. 5,459,127); and L-spermine-5-carboxyl-3-(DL-1,2-dipalmitoyl-dimethylaminopropyl-β-hydroxyethylamine) (SPC-DPRI diester) (see e.g., U.S. Pat. No. 5,459,127).

U.S. Pat. Nos. 5,589,466; 5,693,622; 5,580,859; 5,703,055 and international publication No. WO94/9469 (which are herein incorporated by reference in their entirety) provide methods for delivering DNA-cationic lipid complexes to a cell or mammal.

The choice of exogenous lipid composition will depend on the cell-type to be transduced, since some reagents work better, for example in an immortalized cell line vs. a primary cell. In addition, some transfection reagents work well in the presence of serum, while others require a serum-free media in order to form lipid complexes. The ratio of culture medium to transfection reagent will also vary depending on the amount of nucleocapsid required and the cell to be transduced. It is well within the ability for one of skill in the art to choose an exogenous lipid composition that is appropriate for the system to be used and to employ the methods described herein. It is also contemplated herein that a mixture of more than one lipid is used as an exogenous lipid composition for encapsulating a purified nucleocapsid.

### Formation of Liposomes

Liposomes have been used in the encapsulation of pharmaceuticals or supplements for delivery. The liposomic structure provides drugs a shield from external derogatory factors and can deliver the encapsulated pharmaceutical to cells via endocytosis. Recent advances in nanotechnology and molecular biology have enabled the creation of smart liposomes which can target specific cells/organs and which have programmed release of contents (nucleic acids, drugs etc) based on external or internal cues, e.g., pH, temperature or the presence of a complementary enzyme, among others. Use of liposome technology in terms of the methods described herein relates to the encapsulation, protection from degradation and delivery of a purified nucleocapsid to cells. Liposomes can be engineered such that the molecules are specifically targeted to a particular cell type by recognition of a receptor on the external surface of the cell (as previously disclosed, for example, in U.S. Pat. Nos. 5,258,499 and 6,573,101, which are incorporated herein by reference 92,93).

A variety of methods are available for preparing liposomes as described in; e.g., Szoka et al., Ann. Rev. Biophys. Bioeng. 9:467 (1980), U.S. Pat. Nos. 4,186,183, 4,217,344, 4,235,871, 4,261,975, 4,485,054, 4,501,728, 4,774,085, 4,837,028, 4,946,787, PCT Publication No. WO 91/17424, Szoka & Papahadjopoulos, Proc. Natl. Acad. Sci. USA, 75: 4194-4198 (1978), Deamer and Bangham, Biochim. Biophys. Acta, 443: 629-634 (1976); Fraley, et al., Proc. Natl. Acad. Sci. USA, 76: 3348-3352 (1979); Hope, et al., Biochim. Biophys. Acta, 812: 55-65 (1985); Mayer, et al., Biochim. Biophys. Acta, 858: 161-168 (1986); Williams, et al., Proc. Natl. Acad. Sci., 85: 242-246 (1988), the text Liposomes, Marc J. Ostro, ed., Marcel Dekker, Inc., New York, 1983, Chapter 1, and Hope, et al., Chem. Phys. Lip. 40: 89 (1986), all of which are incorporated herein by reference in their entirety. Suitable methods include, e.g., sonication, extrusion, high pressure/homogenization, microfluidization, detergent dialysis, calcium-induced fusion of small liposome vesicles, and ether-infusion methods. These methods are all known to those of skill in the art. One method produces multilamellar vesicles of heterogeneous sizes. In this method, the vesicle-forming lipids are dissolved in a suitable organic solvent or solvent system and dried under vacuum or an inert gas to form a thin lipid film. If desired, the film may be redissolved in a suitable solvent, such as tertiary butanol, and then lyophilized to form a more homogeneous lipid mixture which is in a more easily hydrated powder-like form. This film is covered with an aqueous buffered solution and allowed to hydrate, typically over a 15-60 minute period with agitation. The size distribution of the resulting multilamellar vesicles can be shifted toward smaller sizes by hydrating the lipids under more vigorous agitation conditions or by adding solubilizing detergents such as deoxycholate.

Unilamellar vesicles are generally prepared by sonication or extrusion. Sonication is generally performed with a tip sonifier, such as a Branson tip sonifier, in an ice bath. Typically, the suspension is subjected to several sonication cycles. Extrusion may be carried out by biomembrane extruders, such as the Lipex Biomembrane Extruder. Defined pore size in the extrusion filters may generate unilamellar liposomal vesicles of specific sizes. The liposomes may also be formed by extrusion through an asymmetric ceramic filter, such as a Ceraflow Microfilter, commercially available from the Norton Company, Worcester Mass.

### Promoters

An exogenous gene to be expressed in a mammalian cell can be operably linked to a “mammalian-active” promoter (i.e., a promoter that directs transcription in a mammalian cell). For example, the mammalian-active viral CMV IE1 promoter permits non-cell specific (i.e., ubiquitous) expression of a heterologous gene. Where cell-type specific expression of the exogenous gene is desired, the exogenous gene can be operably linked to a mammalian-active, cell-type-specific promoter, such as a promoter that is specific for e.g., liver cells, brain cells (e.g., neuronal cells), glial cells, Schwann cells, lung cells, kidney cells, spleen cells, muscle cells, or skin cells. As but one example, consider the instance where liver-specific expression is desired. In this instance, a liver cell-specific promoter can include a promoter of a gene encoding albumin, α-1-antitrypsin, pyruvate kinase, phosphoenol pyruvate carboxykinase, transferrin, transthyretin, α-fetoprotein, α-fibrinogen, or β-fibrinogen. Other examples include several liver-specific promoters, such as the albumin promoter/enhancer, which has been described and can be used to achieve liver-specific expression of the exogenous gene (see, e.g., Shen et al., 1989, DNA 8:101-108; Tan et al., 1991, Dev. Biol. 146:24-37; McGrane et al., 1992, TIBS 17:40-44; Jones et al., J. Biol. Chem. 265:14684-14690; and Shimada et al., 1991, FEBS Letters 279:198-200). Alternatively, a hepatitis virus promoter (e.g., hepatitis A, B, C, or D viral promoter) can be used. If desired, a hepatitis B viral enhancer may be used in conjunction with a hepatitis B viral promoter. Preferably, an albumin promoter and enhancer would be used. As an example, an α-fetoprotein promoter is particularly useful for driving expression of an exogenous gene when it is desired to express a gene for treating a hepatocellular carcinoma. Other preferred liver-specific promoters include promoters of the genes encoding the low density lipoprotein receptor, α2-macroglobulin, μ1-antichymotrypsin, μ2-HS glycoprotein, haptoglobin, ceruloplasmin, plasminogen, complement proteins (C1q, C1r, C2, C3, C4, C5, C6, C8, C9, complement Factor I and Factor H), C3 complement activator, β-lipoprotein, and μ1-acid glycoprotein.

As another example, for expression of an exogenous gene specifically in neuronal cells, a neuron-specific enolase promoter can be used (see Forss-Petter et al., 1990, Neuron 5: 187-197). For expression of an exogenous gene in dopaminergic neurons, a tyrosine hydroxylase promoter can be used. For expression in pituitary cells, a pituitary-specific promoter such as POMC may be useful (Hammer et al., 1990, Mol. Endocrinol. 4:1689-97). Examples of muscle specific promoter include, for example α-myosin heavy chain promoter, and the MCK promoter. Other cell specific promoters active in mammalian cells are also contemplated herein. Such promoters provide a convenient means for controlling expression of the exogenous gene in a cell of a cell culture or within a mammal.

Promoters that are inducible by external stimuli also can be used for driving expression of the exogenous gene. Preferred inducible promoters include enkephalin promoters (e.g., the human enkephalin promoter), metallothionein promoters, mouse mammary tumor virus promoters, promoters based on progesterone receptor mutants, tetracycline-inducible promoters, rapamycin-inducible promoters, and ecdysone-inducible promoters.

The genome of the non-mammalian virus can be engineered to include one or more genetic elements, such as a promoter of a long-terminal repeat of a transposable element or a retrovirus (e.g., Rous Sarcoma Virus); an integrative terminal repeat of an adeno-associated virus; and/or a cell-immortalizing sequence. The genome of the non-mammalian DNA virus used in the invention can include a polyadenylation signal and an RNA splicing signal positioned for proper processing of the product of the exogenous gene. In addition, it is contemplated herein that other signals useful for increasing expression of a protein from the introduced nucleic acid can be incorporated by one of skill in the art, including but not limited to RNA export signals and/or stabilizing sequences (e.g., Woodchuck Hepatitis Virus post-transcriptional response element (WPRE)). Methods for inducing gene expression from each of these promoters are known in the art.

### Modifications of the Artificial Coat: Addition of Coat Proteins and Targeting Moieties

In some cases it may be desirable to add an altered coat protein or targeting moiety to the exogenous lipid composition prior to contacting the lipid and the nucleocapsid. In these cases, the altered coat protein or targeting moiety incorporates into the artificial coat comprised by exogenous lipid, and confers cell-specific targeting of a delivery composition.

By “altered coat protein” is meant any polypeptide that (i) is capable of being or designed to be incorporated into an artificial coat, (ii) is not naturally present on the surface of the non-mammalian DNA virus used to prepare a nucleocapsid delivery composition, and (iii) allows entry to a mammalian cell by binding to the cell and/or facilitating escape from the mammalian endosome into the cytosol of the cell. In the methods described herein, it is contemplated that such an altered coat protein could be added exogenously to the purified nucleocapsids during preparation of a lipid encapsulated nucleocapsid, thus further enhancing mammalian-cell type specificity (e.g., uptake by certain cell types or species; see for example U.S. Pat. Nos. 6,338,953, 6,190,887; and 6,183,993, which are incorporated herein in their entirety).

An altered coat protein can include all or a portion of a coat protein of a “mammalian” virus, i.e., a virus that naturally infects and replicates in a mammalian cell (e.g., an influenza virus). If desired, the altered coat protein can be a “fusion protein,” i.e., an engineered protein that includes part or all of two (or more) distinct proteins derived from one or multiple distinct sources (e.g., proteins of different species). Typically, a fusion protein used in the methods and compositions described herein can include (i) a polypeptide that has a transmembrane region of a transmembrane protein (e.g., baculovirus gp64) fused to (ii) a polypeptide that binds a mammalian cell (e.g., an extracellular domain of VSV-G).

Any transmembrane protein that binds to a target mammalian cell, or that mediates membrane fusion to allow escape from endosomes, is contemplated for use as an altered coat protein on a lipid encapsulated nucleocapsid delivery composition. Preferably, the altered coat protein is the polypeptide (preferably a glycosylated version) of a glycoprotein that naturally mediates viral infection of a mammalian cell (e.g., a coat protein of a mammalian virus, such as a lentivirus, an influenza virus, a hepatitis virus, or a rhabdovirus). Other useful altered coat proteins include proteins that bind to a receptor on a mammalian cell and stimulate endocytosis. Suitable altered coat proteins, which can be derived from viruses such as HIV, influenza viruses, rhabdoviruses, and human respiratory viruses, are discussed in U.S. Pat. Nos. 6,338,953, 6,190,887; and 6,183,993. If desired, more than one coat protein can be used as altered coat proteins. For example, a first altered coat protein may be a transmembrane protein that binds to a mammalian cell, and a second coat protein may mediate membrane fusion and escape from endosomes. Measles virus has two proteins that have been shown to be useful in this regard (see e.g., Funke, S. et al., (2008) Mol Ther. 16(8):1427-36). An altered coat protein can also be used with the methods and compositions described herein to prevent activation of host complement systems. Exemplary proteins useful for preventing complement activation include, but are not limited to CD59 and Decay Accelerating factor (DAF).

If desired, the exogenous lipid of a lipid encapsulated nucleocapsid delivery composition can further comprise a targeting moiety (e.g., ligand) that binds to mammalian cells to facilitate entry. For example, the composition can include as a ligand an asialoglycoprotein that binds to mammalian lectins (e.g., the hepatic asialoglycoprotein receptor), facilitating entry into mammalian cells. Single chain antibodies, which can target particular cell surface markers, are also contemplated herein for use as targeting moieties.

### Genetic Manipulation

The methods described herein involve the physical removal of the outer envelope of a virus particle and subsequent coating of the nucleocapsid with an exogenous or synthetic lipid composition to produce a lipid encapsulated nucleocapsid delivery composition with the ability to transduce a mammalian cell and express an exogenous gene in the mammalian cell. The techniques disclosed herein are especially useful for delivering heterologous genes to mammalian cells that are refractory to conventional vectors.

In contrast to conventional gene expression methods, the methods described herein involve preparing lipid encapsulated nucleocapsids from non-mammalian DNA viruses that do not naturally infect and replicate in mammalian cells. Thus, the methods and compositions described herein are based on the modification of existing properties of a non-mammalian DNA virus that allow it to deliver a gene to a mammalian cell and direct gene expression within the mammalian cell. In contrast, conventional gene therapy vectors require that one disable viral functions, such as expression of viral genes and viral genome replication.

In the present method, the non-mammalian viral nucleocapsid serves as a “shell” for the delivery of DNA to the mammalian cell. The viral DNA is engineered to contain transcriptional control sequences that are active in a mammalian cell, to allow expression of the gene of interest in the target cell. Conventional recombinant DNA techniques can be used for inserting such sequences. Because the lipid encapsulated nucleocapsid composition used in the methods and compositions described herein are produced from non-mammalian viruses that are not capable of replicating in mammalian cells, it is not necessary to delete essential viral functions to render them defective. It is preferred, however, that the virus naturally replicate in a eukaryotic species (e.g., an insect, a plant, or a fungus). Examples of viruses that can be used to produce a lipid encapsulated nucleocapsid capable of expressing an exogenous gene in a mammalian cell in accordance with the methods disclosed herein are discussed in U.S. Pat. Nos. 6,338,953, 6,190,887, 6,183,993, 7,192,933, 6,338,962, 6,281,009, 6,283,914, 5,871,986, 5,731,182, which are incorporated herein in their entirety. Preferably, the genome of the virus used in the methods and compositions described herein is normally transported to the nucleus in its natural host species because nuclear localization signals function similarly in invertebrate and in mammalian cells.

Established methods for manipulating recombinant viruses can be incorporated into these new methods for expressing an exogenous gene in a mammalian cell. For example, viral genes can be deleted from the virus and supplied in trans via packaging lines. Deletion of such genes may be desired in order to (1) suppress expression of viral gene products that might provoke an immune response, (2) provide additional space in the viral vector, or (3) provide additional levels of safety in maintaining the virus in a cell.

Since most promoters of non-mammalian viruses are not active in mammalian cells, the exogenous gene should be operably linked to a promoter that is capable of directing gene transcription in a mammalian cell (i.e., a “mammalian-active” promoter). Examples of suitable promoters include the RSV LTR, the SV40 early promoter, CMV IE promoters (e.g., the human CMV IE1 promoter), the adenovirus major late promoter, and the Hepatitis B viral promoter. Other suitable “mammalian-active” promoters include “mammalian promoters,” i.e., sequences corresponding to promoters that naturally occur in, and drive gene expression in, mammalian cells. Often, “mammalian promoters” are also cell-type-specific, stage-specific, or tissue-specific in their ability to direct transcription of a gene, and such promoters can be used advantageously in the invention as a means for controlling expression of the exogenous gene.

If desired, the cell to be transduced can first be stimulated to be mitotically active. In culture, agents such as chloroform can be used to this effect; in vivo, stimulation of e.g., a liver cell division can be induced by partial hepatectomy (see, e.g., Wilson, et al., 1992, J. Biol. Chem. 267:11283-11489). Optionally, the virus genome can be engineered to carry a herpes simplex virus thymidine kinase gene; this would allow cells harboring the virus genome to be killed by gancicylovir.

If desired, the non-mammalian virus used as a source of nucleocapsid could be engineered such that it is defective in growing on its natural non-mammalian host cell (e.g., insect cell). Such strains of viruses could provide added safety and be propagated on a complementing packaging line. For example, a defective baculovirus could be made in which an immediate early gene, such as IE1, has been deleted. This deletion can be made by targeted recombination in yeast or E. coli, and the defective virus can be replicated in insect cells in which the IE1 gene product is supplied in trans. If desired, the virus can be treated with neuraminidase to reveal additional terminal galactose residues prior to infection (see, e.g., Morell et al., 1971, J. Biol. Chem. 246:1461-1467).

### Therapeutic Genes

A variety of exogenous genes may be used to encode gene products such as proteins, antisense nucleic acids (e.g., RNAs), RNA interference molecules (e.g., siRNA, miRNA, shRNA) or catalytic RNAs. If desired, the gene product (e.g., protein or RNA) can be purified from the cell. Thus, the methods and compositions disclosed herein can be used in the manufacture of a wide variety of proteins that are useful in the fields of biology and medicine. The methods and compositions described herein can also be used to treat a gene deficiency disorder; particularly appropriate genes for expression include those genes which are expressed in normal cells of the type of cell to be transduced, but are expressed at a level that is lower than the normal level for the particular cell to be transduced.

The methods and compositions described herein can be used to express various “therapeutic” genes in a cell. A “therapeutic” gene is one that, when expressed, confers a beneficial effect on the cell or tissue in which it is present, or on a mammal in which the gene is expressed. Examples of “beneficial effects” include amelioration of a sign or symptom of a condition or disease, prevention or inhibition of a condition or disease, or conferral of a desirable characteristic, including even temporary amelioration of signs or symptoms of a disorder. Included among the therapeutic genes are those genes that correct a gene deficiency disorder in a cell or mammal (correction of a disorder need not be equivalent to curing a patient suffering from a disorder). Also included are genes that are expressed in one cell, yet which confer a beneficial effect on a second cell. For example, a gene encoding insulin can be expressed in a pancreatic cell from which the insulin is then secreted to exert a beneficial effect on other cells of the mammal. Other therapeutic genes include sequences that are transcribed into RNAs (e.g., antisense or RNA interference-type molecules) that inhibit transcription or translation of a mutant gene or a gene that is expressed at undesirably high levels. For example, an antisense or RNA interference molecule expressing gene or construct that inhibits expression of a gene encoding an oncogenic protein is considered a therapeutic gene.

The methods and compositions described herein can be used to express a therapeutic gene in order to treat a disorder (e.g., a gene deficiency disorder). Some non-limiting examples of genes involved in a gene deficiency disorder include carbamoyl synthetase I, ornithine transcarbamylase, arginosuccinate synthetase, arginosuccinate lyase, and arginase. Other desirable gene products include, but are not limited to, fumarylacetoacetate hydrolase, phenylalanine hydroxylase, alpha-1 antitrypsin, glucose-6-phosphatase, low-density-lipoprotein receptor, porphobilinogen deaminase, factor VIII, factor IX, cystathione β-synthase, branched chain ketoacid decarboxylase, albumin, isovaleryl-CoA dehydrogenase, propionyl CoA carboxylase, methyl malonyl CoA mutase, glutaryl CoA dehydrogenase, insulin, β-glucosidase, pyruvate carboxylase, hepatic phosphorylase, phosphorylase kinase, glycine decarboxylase (also referred to as P-protein), H-protein, T-protein, Menkes disease copper-transporting ATPase, and Wilson's disease copper-transporting ATPase. Other examples of desirable genes for expression with the invention include genes encoding tumor suppressors (e.g., p53), or CFTR (e.g., for treating cystic fibrosis).

By “antisense” nucleic acid is meant a nucleic acid molecule (i.e., RNA) that is complementary (i.e., able to hybridize in vivo or under stringent in vitro conditions) to all or a portion of a target nucleic acid (e.g., a gene or mRNA) that encodes a polypeptide of interest. If desired, conventional methods can be used to produce an antisense nucleic acid that contains desirable modifications. For example, a phosphorothioate oligonucleotide can be used as the antisense nucleic acid in order to inhibit degradation of the antisense oligonucleotide by nucleases in vivo. Where the antisense nucleic acid is complementary to only a portion of the target nucleic acid encoding the polypeptide to be inhibited, the antisense nucleic acid should hybridize close enough to some critical portion of the target nucleic acid (e.g., in the translation control region of the non-coding sequence, or at the 5′ end of the coding sequence) such that it inhibits translation of a functional polypeptide (i.e., a polypeptide that carries out an activity that one wishes to inhibit (e.g., an enzymatic activity)). Typically, this means that the antisense nucleic acid should be complementary to a sequence that is within the 5′ half or third of a target mRNA to which the antisense nucleic acid hybridizes. As used herein, an “antisense gene” is a nucleic acid that is transcribed into an antisense RNA. Typically, such an antisense gene includes all or a portion of the target nucleic acid, but the antisense gene is operably linked to a promoter such that the orientation of the antisense gene is opposite to the orientation of the sequence in the naturally-occurring gene.

RNA interference agents can be used with the methods described herein, to inhibit the expression and/or activity of a polypeptide. “RNA interference (RNAi)” is an evolutionarily conserved process whereby the expression or introduction of RNA of a sequence that is identical or highly similar to a target gene results in the sequence specific degradation or specific post-transcriptional gene silencing (PTGS) of messenger RNA (mRNA) transcribed from that targeted gene (see Coburn, G. and Cullen, B., J. of Virology 76(18):9225 (2002), herein incorporated by reference in its entirety), thereby inhibiting expression of the target gene. As used herein, “inhibition of target gene expression” includes any decrease in expression or protein activity or level of the target gene or protein encoded by the target gene as compared to a situation wherein no RNA interference has been induced. The decrease can be of at least 20%, 30%, 40%, 50%, 60%, 70%, 80%, 90%, 95% or 99% or more as compared to the expression of a target gene or the activity or level of the protein encoded by a target gene which has not been targeted by an RNA interfering agent. RNA interfering agents contemplated for use with the methods described herein include, but are not limited to, siRNA, shRNA, miRNA, and dsRNA.

The target gene or sequence of the RNA interfering agent can be a cellular gene or genomic sequence. An siRNA can be substantially homologous to the target gene or genomic sequence, or a fragment thereof. As used in this context, the term “homologous” is defined as being substantially identical, sufficiently complementary, or similar to the target mRNA, or a fragment thereof, to effect RNA interference of the target. Preferably, the siRNA is identical in sequence to its target and targets only one sequence. Each of the RNA interfering agents, such as siRNAs, can be screened for potential off-target effects by, for example, expression profiling. Such methods are known to one skilled in the art and are described, for example, in Jackson et al., Nature Biotechnology 6:635-637 (2003), herein incorporated by reference in its entirety.

It is well within the ability of one skilled in the art to design and test for siRNAs that are useful for inhibiting expression and/or activity of a gene or gene product. It is important to note that double-stranded siRNA or shRNA molecules that are cleaved by Dicer in the cell can be up to 100 times more potent than a 21-mer siRNA or shRNA molecule supplied exogenously (Kim, D H., et al (2005) Nature Biotechnology 23(2):222-226). Thus, an RNAi molecule can be designed to be more effective by providing a sequence for Dicer cleavage. Methods for effective siRNA design for use in vivo can be found in U.S. Pat. No. 7,427,605, which is herein incorporated by reference in its entirety.

### Cells

Essentially any mammalian cell can be targeted for transduction by the lipid encapsulated nucleocapsid delivery compositions as described herein. However, it is preferred that the mammalian cell is a human cell. The cell can be a primary cell (e.g., a primary hepatocyte, primary neuronal cell, or primary myoblast) or it can be a cell of an established cell line. It is not necessary that the cell be capable of undergoing cell division; a terminally differentiated cell can be used in the methods described herein. If desired, the nucleocapsid delivery composition can be introduced into a primary cell approximately 24 hours after plating of the primary cell to maximize the efficiency of transduction. Some examples of mammalian cells include, but are not limited to the following: a liver-derived cell or cell line, such as a HepG2 cell, a Hep3B cell, a Huh-7 cell, an FTO2B cell, a Hepa1-6 cell, or an SK-Hep-1 cell) or a Kupffer cell; a kidney cell or cell line, such as a cell of the kidney cell line 293, a PC12 cell (e.g., a differentiated PC12 cell induced by nerve growth factor), a COS cell (e.g., a COS7 cell), or a Vero cell (an African green monkey kidney cell); a neuronal cell or cell line, such as a fetal neuronal cell, cortical pyramidal cell, mitral cell, a granule cell, or a brain cell (e.g., a cell of the cerebral cortex; an astrocyte; a glial cell; a Schwann cell); a muscle cell or cell line, such as a myoblast or myotube (e.g., a C₂C₁₂ cell); an embryonic stem cell or cell line, a spleen cell or cell line (e.g., a macrophage or lymphocyte); an epithelial cell or cell line, such as a HeLa cell (a human cervical carcinoma epithelial line); a fibroblast cell or cell line, such as an NIH3T3 cell; an endothelial cell or cell line; a WISH cell; an A549 cell; or a bone marrow stem cell or cell line. Other potentially useful mammalian cells include CHO/dhfr⁻ cells, Ramos, Jurkat, HL60, and K-562 cells.

Where the cell is maintained under in vitro conditions, conventional tissue culture conditions and methods can be used. In some embodiments, the cell can be maintained on a substrate that contains collagen, such as, for example, Type I collagen or rat tail collagen, fibronectin, or a matrix containing laminin. As an alternative to, or in addition to, maintaining the cell under in vitro conditions, the cell can be maintained under in vivo conditions (e.g., in a human). Implantable versions of collagen substrates are also suitable for maintaining the transduced cells under in vivo conditions in practicing the invention (see, e.g., Hubbell et al., 1995, Bio/Technology 13:565-576 and Langer and Vacanti, 1993, Science 260: 920-925).

### Therapeutic Uses

The compositions and methods described herein further feature a method for treating disease in a mammal, for example introducing into a cell of the mammal a lipid encapsulated nucleocapsid delivery composition, the nucleic acid of which encodes and directs the expression of a therapeutic gene.

In one embodiment the disease to be treated is cancer. In the treatment of cancer, exemplary genes to be expressed can include, for example, a cytotoxic factor or a factor that renders cells expressing it susceptible to killing with a drug (e.g., thymidine kinase, diphtheria toxin chimera, or cytosine deaminase). The exogenous gene can be expressed in a variety of cells, e.g., hepatocytes; cells of the central nervous system, including neural cells such as neurons from brain, spinal cord, or peripheral nerve; adrenal medullary cells; glial cells; skin cells; spleen cells; muscle cells; kidney cells; and bladder cells. Thus, the methods and compositions described herein can be used to treat various diseases including cancerous or non-cancerous tumors, including carcinomas (e.g., hepatocellular carcinoma), sarcomas, gliomas, and neuromas. Either in vivo or in vitro methods can be used to introduce the nucleocapsid delivery composition into the cell in this aspect of the invention. Preferably, the exogenous gene is operably linked to a promoter that is active in cancerous cells, but not in other cells, of the mammal. For example, the α-fetoprotein promoter is active in cells of hepatocellular carcinomas and in fetal tissue but it is otherwise not active in mature tissues.

Another example of a disease that can be treated with the compositions and methods described herein is a neurological disorder (e.g., Parkinson's Disease, Alzheimer's Disease, or disorders resulting from injuries to the central nervous system) in a mammal. The method involves (a) contacting a cell with a lipid encapsulated nucleocapsid delivery composition, the nucleic acid of which includes an exogenous gene encoding a therapeutic protein, and (b) maintaining the cell under conditions such that the exogenous gene is expressed in the mammal. Particularly useful exogenous genes include those that encode therapeutic proteins such as nerve growth factor, hypoxanthine guanine phosphoribosyl transferase (HGPRT), tyrosine hydroxylase, dopadecarboxylase, brain-derived neurotrophic factor, basic fibroblast growth factor, sonic hedgehog protein, glial derived neurotrophic factor (GDNF) and RETLI (also known as GDNFRα, GFR-1, and TRN1). Both neuronal and non-neuronal cells (e.g., fibroblasts, myoblasts, and kidney cells) are useful in this aspect of the invention. Such cells can be autologous or heterologous to the treated mammal. Preferably, the cell is autologous to the mammal, as such cells obviate concerns about graft rejection. Preferably, the cell is a primary cell, such as a primary neuronal cell or a primary myoblast.

In addition to these examples, the treatment of obesity, diabetes, metabolic disorders, autoimmune diseases, cancer, cardiovascular disease, kidney disorders, hematological disorders, neurological disorders, retinopathies, gastrointestinal disorders, and liver disease, among others, is also contemplated herein. As well, numerous disorders are known to be caused by single gene defects, and many of the genes involved in gene deficiency disorders have been identified and cloned. Using standard cloning techniques (see, e.g., Ausbel et al., Current Protocols in Molecular Biology, John Wiley & Sons, (1989)), a non-mammalian virus genome can be engineered to encode and package a desired exogenous gene in a mammalian cell (e.g., a human cell).

The compositions and methods described herein can be used to facilitate the expression of a desired gene in a cell having no obvious deficiency. For example, the invention can be used to express insulin in a hepatocyte of a patient in order to supply the patient with insulin in the body. Other examples of proteins which can be expressed in a mammalian cell (e.g., a liver cell) for delivery into the system circulation of the mammal include hormones, growth factors, cytokines and interferons. The methods and compositions described herein can also be used to express a regulatory gene or a gene encoding a transcription factor (e.g., a VP16-tet repressor gene fusion) in a cell to control the expression of another gene (e.g., genes which are operably-linked to a tet operator sequence; see, e.g., Gossen et al., 1992, PNAS 89:5547-5551). If desired, a tumor suppressor gene, such as the gene encoding p53, can be expressed in a cell in a method of treating cancer.

Other useful gene products include RNA molecules for use in RNA decoy, antisense, or ribozyme-based methods of inhibiting gene expression (see, e.g., Yu et al., 1994, Gene Therapy 1:13-26). If desired, the methods and compositions described herein can be used to express a gene, such as cytosine deaminase, whose product will alter the activity of a drug or prodrug, such as 5-fluorocytosine, in a cell (see, e.g., Harris et al., 1994, Gene Therapy 1: 170-175). Methods such as the use of ribozymes, antisense RNAs, transdominant repressors, polymerase mutants, or core or surface antigen mutants can be used to suppress viruses, e.g., hepatitis viruses (e.g., hepatitis virus A, B, C, or D) in a cell. Other disorders such as familial hemachromatosis can also be treated with the invention by treatment with the normal version of the affected gene.

The therapeutic effectiveness of expressing an exogenous gene in a cell can be assessed by monitoring the patient for known signs or symptoms of a disorder. Parameters for assessing treatment methods are known to those skilled in the art of medicine (see, e.g., Maestri et al., 1991, J. Pediatrics, 119:923-928).

In one approach for the methods described herein, the lipid encapsulated nucleocapsid can be used to transduce a cell outside of the mammal to be treated (e.g., a cell in a donor mammal or a cell in vitro), and the transduced cell can then be administered to the mammal to be treated. In this method, the cell can be autologous or heterologous to the mammal to be treated. Again taking expression in the liver as an example, an autologous hepatocyte obtained in a liver biopsy can be used (see, e.g., Grossman et al., 1994, Nature Genetics 6:335). The cell can then be administered to the patient by injection (e.g., into the portal vein). In such a method, a volume of hepatocytes totaling about 1%-10% of the volume of the entire liver is preferred. Where the viruses described herein are used to express an exogenous gene in a liver cell, the liver cell can be delivered to the spleen, and the cell can subsequently migrate to the liver in vivo (see, e.g., Lu et al., 1995, Hepatology 21:7752-759). If desired, the nucleocapsid delivery composition may be delivered to a cell by employing conventional techniques for perfusing fluids into organs, cells, or tissues (including the use of infusion pumps and syringes). For perfusion, the nucleocapsid delivery composition is generally administered at a titer of 1×10⁶ to 1×10¹⁰ particles/ml (preferably 1×10⁹ to 1×10¹⁰ particles/ml) in a volume of 1 to 500 ml, over a time period of 1 minute to 6 hours. If desired, multiple doses of the lipid encapsulated nucleocapsid can be administered to a patient intravenously for several days in order to increase the level of expression as desired. Similar approaches can be taken for other tissues, the practitioner being mindful of specific characteristics of the target tissue to ensure infection and expression in that tissue.

The optimal amount of a nucleocapsid delivery composition or number of transduced cells to be administered to a mammal and the frequency of administration are dependent upon factors such as the sensitivity of methods for detecting expression of the exogenous gene, the strength of the promoter used, the severity of the disorder to be treated, and the target cell(s) of the nucleocapsid delivery composition. Generally, the delivery composition is administered at a multiplicity of “infection” of about 0.1 to 1,000; preferably, the multiplicity of “infection” is about 5 to 100; more preferably, the multiplicity of “infection” is about 10 to 50.

If desired, the invention can be used to express a dominant negative mutant in a mammalian cell. For example, viral assembly in a cell can be inhibited or prevented by expressing in that cell a dominant negative mutant of a viral capsid protein (see, e.g., Scaglioni et al., 1994, Virology 205:112-120; Scaglioni et al., 1996, Hepatology 24:1010-1017; and Scaglioni et al., 1997, J. Virol. 71:345-353).

If desired, the nucleic acid delivery compositions described herein can be used to propagate genetic constructs in non-mammalian (e.g., insect) cells, with the advantage of inhibiting DNA methylation of the product. It has been observed that a promoter may become methylated in cell lines or tissues in which it is not normally expressed, and that such methylation is inhibitory to proper tissue specific expression (Okuse et al., 1997, Brain Res. Mol. Brain Res. 46:197-207; Kudo et al., 1995, J. Biol. Chem. 270:13298-13302). For example, a neural promoter may become methylated in a non-neural mammalian cell. By using, for example, insect cells (e.g., Sf9 cells) to propagate a baculovirus carrying an exogenous gene and a mammalian promoter (e.g., a neural promoter), the methods and compositions described herein provide a means for inhibiting DNA methylation of the promoter prior to administration of the exogenous gene to the mammalian cell in which the exogenous gene will be expressed (e.g., a neural cell).

### Delivery Methods

The lipid encapsulated nucleocapsid composition can be introduced into a cell in vitro or in vivo. Where the lipid encapsulated nucleocapsid delivery composition is introduced into a cell in vitro, the transduced cell can subsequently be introduced into a mammal, if desired. Similarly, where the lipid encapsulated nucleocapsids described herein are used to express an exogenous gene in more than one cell, a combination of in vitro and in vivo methods may be used to introduce the gene into more than one mammalian cell.

If desired, the nucleocapsid delivery composition can be introduced into the cell by administering the delivery composition to a mammal that carries the cell. For example, the lipid encapsulated nucleocapsid can be administered to a mammal by subcutaneous, intravenous, or intraperitoneal injection. If desired, a slow-release device, such as an implantable pump, may be used to facilitate delivery of the nucleocapsid delivery composition to cells of the mammal. A particular cell type within the mammal can be targeted by modulating the amount of the delivery composition administered to the mammal and by controlling the method of delivery. For example, intravascular administration of the lipid encapsulated nucleocapsid composition to the portal, splenic, or mesenteric veins or to the hepatic artery may be used to facilitate targeting of the nucleocapsid delivery composition to liver cells. In another method, the composition may be administered to cells or an organ of a donor individual (human or non-human) prior to transplantation of the cells or organ to a recipient.

The lipid encapsulated nucleocapsid can be formulated into a pharmaceutical composition by admixture with a pharmaceutically acceptable non-toxic excipient or carrier (e.g., saline) for administration to a mammal. In practicing the invention, the composition can be prepared for use in parenteral administration (e.g., for intravenous injection (e.g., into the portal vein)), intra-arterial injection (e.g., into the femoral artery or hepatic artery), intraperitoneal injection, intrathecal injection, or direct injection into a tissue or organ (e.g., intramuscular injection). In particular, the lipid encapsulated nucleocapsid delivery composition can be prepared in the form of liquid solutions or suspensions in conventional excipients. The composition can also be prepared for intranasal or intrabronchial administration, particularly in the form of nasal drops or aerosols in conventional excipients. If desired, the composition can be sonicated in order to minimize clumping of the lipid encapsulated nucleocapsid delivery composition in preparing the delivery composition.

In a preferred method of administration, the composition is administered to a tissue or organ containing the targeted cells of the mammal. Such administration can be accomplished by injecting a solution containing the lipid encapsulated nucleocapsid into a tissue, such as skin, brain (e.g., the cerebral cortex), kidney, bladder, liver, spleen, muscle, thyroid, thymus, lung, or colon tissue. Alternatively, or in addition, administration can be accomplished by perfusing an organ with a solution containing the delivery composition, according to conventional perfusion protocols.

In another preferred method, the delivery composition is administered intranasally, e.g., by applying a solution of the lipid encapsulated nucleocapsid delivery composition to the nasal mucosa of a mammal. This method of administration can be used to facilitate retrograde transportation of the delivery composition into the brain. This method thus provides a means for delivering the composition to brain cells, (e.g., mitral and granule neuronal cells of the olfactory bulb) without subjecting the mammal to surgery. In an alternative method for using the composition to express an exogenous gene in the brain, the composition is delivered to the brain by osmotic shock according to conventional methods for inducing osmotic shock.

### Detection of Transduction and Gene Expression

Delivery of a lipid encapsulated nucleocapsid to a cell and expression of the exogenous gene can be monitored using standard techniques. For example, delivery of an exogenous gene to a cell can be measured by detecting viral DNA or RNA (e.g., by Southern or Northern blotting, slot or dot blotting, or in situ hybridization, with or without amplification by PCR) or by detecting the expression of a transgene carried by the nucleocapsid delivery composition. Suitable probes which hybridize to nucleic acids of the composition, regulatory sequences (e.g., the promoter), or the exogenous gene can be conveniently prepared by one skilled in the art of molecular biology. Where the invention is used to express an exogenous gene in a cell in vivo, delivery of the lipid encapsulated nucleocapsid to the cell can be detected by obtaining the cell in a biopsy. For example, where the invention is used to express a gene in a liver cell(s), a liver biopsy can be performed, and conventional methods can be used to detect the lipid encapsulated nucleocapsid in a cell of the liver.

Expression of an exogenous gene in a cell of a mammal can also be followed by assaying a cell or fluid (e.g., serum) obtained from the mammal for RNA or protein corresponding to the gene. Detection techniques commonly used by molecular biologists (e.g., Northern or Western blotting, in situ hybridization, slot or dot blotting, PCR amplification, SDS-PAGE, immunostaining, RIA, and ELISA) can be used to measure gene expression. If desired, a reporter gene (e.g., lacZ) can be used to measure the ability of a lipid encapsulated nucleocapsid to target gene expression to certain tissues or cells. Examination of tissue can involve: (a) snap-freezing the tissue in isopentane chilled with liquid nitrogen; (b) mounting the tissue on cork using O.C.T. and freezing; (c) cutting the tissue on a cryostat into 10 μm sections; (d) drying the sections and treating them with paraformaldehyde; (e) staining the tissue with X-gal (0.5 mg/ml)/ferrocyanide (35 mM)/ferricyanide (35 mM) in PBS; and (f) analyzing the tissue by microscopy.

Even when exposure of cells to the lipid encapsulated nucleocapsid results in expression of the exogenous gene in a relatively low percentage of the cells (in vitro or in vivo), the methods and compositions described herein can be used to identify or confirm the cell- or tissue-type specificity of the promoter which drives expression of the exogenous gene (e.g., a reporter gene such as a chloramphenicol acetyltransferase gene, an alkaline phosphatase gene, a luciferase gene, or a green fluorescent protein gene). Once identified, such a promoter may be employed in any of the conventional methods of gene expression. Similarly, where so desired, only relatively low levels of expression are necessary for provoking an immune response (i.e., produce antibodies) in a mammal against the heterologous gene product. Thus, the gene expression method of the methods described herein can be used in the preparation of antibodies against a preferred heterologous antigen by expressing the antigen in a cell of a mammal. Such antibodies may be used inter alia to purify the heterologous antigen. The gene expression method may also be used to elicit an immuno-protective response in a mammal (i.e., be used as a vaccine) against a heterologous antigen. In addition, the methods and compositions described herein can be used to make a permanent cell line from a cell in which the lipid encapsulated nucleocapsid mediated expression of a cell-immortalizing sequence (e.g., SV40 T antigen).

### Recombinant Protein Isolation

Methods for isolating protein will vary depending on the characteristics of the recombinant protein expressed in a mammalian expression system as described herein. Various methods for protein isolation are well within the abilities for one of skill in the art.

Some non-limiting examples of protein isolation methods include immunoprecipitation, affinity chromatography, column chromatography and fractionation, ammonium sulfate precipitation and other fractionation methods. One of skill in the art would be able to practice the methods described herein for the purpose of recombinant protein production.

It is understood that the foregoing detailed description and the following examples are illustrative only and are not to be taken as limitations upon the scope of the invention. Various changes and modifications to the disclosed embodiments, which will be apparent to those skilled in the art, may be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

The present invention may be as defined in any one of the following numbered paragraphs.
1. A method for preparing a nucleic acid delivery composition, the method comprising contacting a purified nucleocapsid with a lipid, wherein the purified nucleocapsid is isolated from a non-mammalian virus and wherein the outer envelope of the non-mammalian virus has been removed prior to the contacting, whereby a lipid encapsulated nucleocapsid delivery composition capable of delivering a nucleic acid to a mammalian cell is produced.
2. The method of paragraph 1, wherein the non-mammalian virus is an insect virus.
3. The method of paragraph 1 or 2, wherein the non-mammalian virus is a baculovirus.
4. The method of paragraph 1, 2, or 3, wherein the nucleocapsid comprises a nucleic acid encoding a heterologous gene.
5. The method of paragraph 4, wherein the nucleic acid encoding a heterologous gene further comprises a mammalian promoter.
6. The method of any one of paragraphs 1-5, wherein the outer envelope is removed by contacting the isolated non-mammalian virus with an organic solvent.
7. The method of any one of paragraphs 1-6, wherein the contacting the purified nucleocapsid with the lipid comprises sonicating the purified nucleocapsid in the presence of the lipid.
8. A method for producing a recombinant protein of interest, the method comprising the steps of:

(a) contacting a mammalian cell with a lipid encapsulated nucleocapsid delivery composition, wherein the nucleocapsid delivery composition comprises a nucleic acid encoding a recombinant protein of interest and the contacting results in transduction of the cell with the lipid encapsulated nucleocapsid delivery composition;

(b) incubating the mammalian cell under conditions sufficient to permit expression of the recombinant protein.
9. The method of paragraph 8, wherein the recombinant protein is further isolated from the mammalian cell.
10. The method of paragraph 8 or 9, wherein the recombinant protein is a therapeutic protein.
11. The method of paragraph 9, wherein the mammalian cell is comprised by a mammal.
12. The method of paragraph 11, wherein the mammal is a human.
13. The method of any one of paragraphs 8-12, wherein the lipid encapsulated nucleocapsid delivery composition is produced from an insect virus.
14. The method of any one of paragraphs 8-13, wherein the lipid encapsulated nucleocapsid delivery composition is produced from a baculovirus.
15. A method for introducing a nucleic acid to a mammalian cell, the method comprising contacting a mammalian cell with a lipid encapsulated nucleocapsid delivery composition, wherein the nucleocapsid delivery composition comprises a nucleic acid encoding a recombinant protein of interest, and wherein the contacting results in transduction of the mammalian cell with the nucleocapsid delivery composition.
16. The method of paragraph 15, wherein the lipid encapsulated nucleocapsid delivery composition is produced from an insect virus.
17. The method of paragraph 15 or 16, wherein the lipid encapsulated nucleocapsid delivery composition is produced from a baculovirus.
18. A method for introducing a nucleic acid to a mammalian cell, the method comprising contacting a mammalian cell with a lipid encapsulated nucleocapsid delivery composition, wherein the nucleocapsid delivery composition is produced by the steps of:

(a) removing the outer envelope of an isolated non-mammalian virus to produce a purified nucleocapsid, and

(b) contacting the purified nucleocapsid with a lipid,
wherein steps (a) and (b) produce a lipid encapsulated nucleocapsid delivery composition capable of delivering a nucleic acid to a mammalian cell.
19. The method of paragraph 18, wherein the mammalian cell is comprised by a mammal.
20. The method of paragraph 19, wherein the mammal is a human.
21. The method of paragraph 18, 19, or 20, wherein the lipid encapsulated nucleocapsid delivery composition comprises a nucleic acid encoding a protein of interest.
22. The method of any one of paragraphs 18-21, wherein the protein of interest comprises a therapeutic protein.
23. The method of any one of paragraphs 18-22, wherein the non-mammalian virus is an insect virus.
24. The method of any one of paragraphs 18-23, wherein the non-mammalian virus is a baculovirus.
25. A lipid encapsulated nucleocapsid delivery composition, the composition produced by the steps of:
   (a) removing the outer envelope of an isolated non-mammalian virus to produce a purified nucleocapsid, and

(b) contacting the nucleocapsid with a lipid,
wherein steps (a) and (b) produce a lipid encapsulated nucleocapsid delivery composition capable of delivering a nucleic acid to a mammalian cell.
26. The delivery composition of paragraph 25, wherein the non-mammalian virus is an insect virus.
27. The delivery composition of paragraph 25 or 26, wherein the non-mammalian virus is a baculovirus.
28. A lipid encapsulated nucleocapsid delivery composition, the composition comprising a purified nucleocapsid isolated from a non-mammalian virus, and an exogenous lipid composition.
29. The nucleocapsid delivery composition of paragraph 28, wherein the lipid composition is physically associated with the purified nucleocapsid.
30. The nucleocapsid delivery composition of paragraph 28 or 29, wherein the lipid composition encapsulates the purified nucleocapsid.
31. The nucleocapsid delivery composition of paragraph 28, 29, or 30, further comprising a nucleic acid encoding a protein of interest.
32. The nucleocapsid delivery composition of any one of paragraphs 28-31, further comprising a targeting moiety that directs the nucleocapsid delivery composition to a desired mammalian target cell.
33. The nucleocapsid delivery composition of any one of paragraphs 28-32, wherein the non-mammalian virus is an insect virus.
34. The nucleocapsid delivery composition of any one of paragraphs 28-33, wherein the non-mammalian virus is a baculovirus.
35. Use of a lipid encapsulated nucleocapsid delivery composition, comprising a purified nucleocapsid and an exogenous lipid composition, for promoting gene expression in a cell.
36. The use of paragraph 35, wherein the lipid composition is physically associated with the purified nucleocapsid.
37. The use of paragraph 35 or 36, wherein the lipid composition encapsulates the purified nucleocapsid.
38. The use of paragraph 35, 36, or 37, wherein the nucleocapsid delivery composition comprises a nucleic acid encoding a protein of interest.
39. The use of any one of paragraphs 35-38, wherein the nucleocapsid delivery composition further comprises a targeting moiety that directs the nucleocapsid delivery composition to a desired mammalian target cell.
40. The use of any one of paragraphs 35-39, wherein the nucleocapsid delivery composition is derived from an insect virus.
41. The use of any one of paragraphs 35-40, wherein the nucleocapsid delivery composition is derived from a baculovirus.
42. Use of a lipid encapsulated nucleocapsid delivery composition comprising a purified nucleocapsid and an exogenous lipid composition, in the preparation of a medicament for promoting gene expression in a cell.
43. The use of paragraph 42, wherein the lipid composition is physically associated with the purified nucleocapsid.
44. The use of paragraph 42 or 43, wherein the lipid composition encapsulates the purified nucleocapsid.
45. The use of paragraph 42, 43, or 44, wherein the nucleocapsid delivery composition further comprises a nucleic acid encoding a protein of interest.
46. The use of any one of paragraphs 42-45, wherein the nucleocapsid delivery composition further comprises a targeting moiety that directs the nucleocapsid delivery composition to a desired mammalian target cell.
47. The use of any one of paragraphs 42-46, wherein the nucleocapsid delivery composition is produced from an insect virus.
48. The use of any one of paragraphs 42-47, wherein the nucleocapsid delivery composition is produced from a baculovirus.
49. A liposome encapsulated nucleocapsid composition, the composition comprising a purified nucleocapsid isolated from a non-mammalian virus encapsulated in a liposome.
50. A method for preparing a liposome encapsulated nucleocapsid delivery composition, the method comprising:

(a) removing the outer envelope of an isolated non-mammalian virus to produce a purified nucleocapsid;

(b) contacting the nucleocapsid with a lipid to form a mixture; and

(c) treating the mixture to induce liposome formation,
wherein the nucleocapsid is encapsulated by the liposome and wherein the liposome encapsulated nucleocapsid is capable of delivering a nucleic acid to a mammalian cell.

### EXAMPLES

### Example 1

### One Embodiment of the Methods Described Herein

Disclosed herein in Example 1 is a method of preparing a lipid encapsulated nucleocapsid composition comprising a mammalian-active promoter to increase the tropism of gene delivery to transduce a wider variety of mammalian cells than the corresponding unmodified vectors. Example 1 also describes lipid encapsulated nucleocapsid delivery compositions with synthetic lipid surfaces derived from this method.

The method consists of: (1) removal of the existing lipid envelope of a baculovirus comprising a mammalian active promoter, and (2) re-coating the baculovirus nucleocapsid with a artificial viral coat consisting of synthetic lipids.

The first step in the method is to isolate and concentrate baculoviruses containing mammalian-active promoters through conventional methods, such as those described in Boyce, F. M., and Franco, E. A. (2000) Viral Vectors: Basic Science and Gene Therapy. Eaton Publishing, pp 359-367, which is incorporated herein by reference.

The purified baculoviruses are re-suspended in a buffered saline solution such as phosphate buffered saline. The existing lipid envelope of the baculovirus is removed by extraction with an equal volume of an organic solvent such as ethyl ether. The admixture is vortexed well to mix the components and then spun in a centrifuge (15,000 g for 5 min) to separate the aqueous and organic phases. In the case of ethyl ether, the endogenous lipids from the baculovirus coat will be dissolved into the upper ether phase, whilst the baculovirus nucleocapsids will be present at the interface between the two layers. The purified nucleocapsids can then be mechanically removed from the interface and mixed with buffered saline such as phosphate buffered saline. One-fifth volume of a solution of synthetic lipids such as Lipofectamine 2000™ is then added. The nucleocapsid/lipid mixture is then sonicated to allow coating of the nucleocapsids with the lipids. The resulting mixture can be added to mammalian cells to facilitate entry of the nucleocapsid into the cytosol of the target cell and subsequent expression of the mammalian-active promoter in the target cell.

1. A method for preparing a nucleic acid delivery composition, the method comprising contacting a purified nucleocapsid with a lipid, wherein said purified nucleocapsid is isolated from a non-mammalian virus and wherein the outer envelope of said non-mammalian virus has been removed prior to said contacting, whereby a lipid encapsulated nucleocapsid delivery composition capable of delivering a nucleic acid to a mammalian cell is produced.

2. The method of claim 1, wherein said non-mammalian virus is an insect virus.

3. The method of claim 1, wherein said non-mammalian virus is a baculovirus.

4. The method of claim 1, wherein said nucleocapsid comprises a nucleic acid encoding a heterologous gene.

5. The method of claim 4, wherein said nucleic acid encoding a heterologous gene further comprises a mammalian promoter.

6. The method of claim 1, wherein said outer envelope is removed by contacting said isolated non-mammalian virus with an organic solvent.

7. The method of claim 1, wherein said contacting said purified nucleocapsid with said lipid comprises sonicating said purified nucleocapsid in the presence of said lipid.

8. A method for producing a recombinant protein of interest, the method comprising the steps of:
(a) contacting a mammalian cell with a lipid encapsulated nucleocapsid delivery composition, wherein said nucleocapsid delivery composition comprises a nucleic acid encoding a recombinant protein of interest and said contacting results in transduction of said cell with said lipid encapsulated nucleocapsid delivery composition;
(b) incubating said mammalian cell under conditions sufficient to permit expression of said recombinant protein.

9. The method of claim 8, wherein said recombinant protein is further isolated from said mammalian cell.

10. (canceled)

11. The method of claim 9, wherein said mammalian cell is comprised by a mammal.

12. The method of claim 11, wherein said mammal is a human.

13. (canceled)

14. The method of claim 8, wherein said lipid encapsulated nucleocapsid delivery composition is produced from a baculovirus.

15. A method for introducing a nucleic acid to a mammalian cell, the method comprising contacting a mammalian cell with a lipid encapsulated nucleocapsid delivery composition, wherein said nucleocapsid delivery composition comprises a nucleic acid encoding a recombinant protein of interest, and wherein said contacting results in transduction of said mammalian cell with said nucleocapsid delivery composition.

16. (canceled)

17. The method of claim 15, wherein said lipid encapsulated nucleocapsid delivery composition is produced from a baculovirus.

18. (canceled)

19. (canceled)

20. (canceled)

21. (canceled)

22. (canceled)

23. (canceled)

24. (canceled)

25. A lipid encapsulated nucleocapsid delivery composition, the composition produced by the steps of:
(a) removing the outer envelope of an isolated non-mammalian virus to produce a purified nucleocapsid, and
(b) contacting said nucleocapsid with a lipid,
wherein steps (a) and (b) produce a lipid encapsulated nucleocapsid delivery composition capable of delivering a nucleic acid to a mammalian cell.

26. (canceled)

27. The delivery composition of claim 25, wherein said non-mammalian virus is a baculovirus.

28. A lipid encapsulated nucleocapsid delivery composition, the composition comprising a purified nucleocapsid isolated from a non-mammalian virus, and an exogenous lipid composition.

29. (canceled)

30. The nucleocapsid delivery composition of claim 28, wherein said lipid composition encapsulates said purified nucleocapsid.

31. (canceled)

32. The nucleocapsid delivery composition of claim 28, further comprising a targeting moiety that directs said nucleocapsid delivery composition to a desired mammalian target cell.

33. (canceled)

34. The nucleocapsid delivery composition of claim 28, wherein said non-mammalian virus is a baculovirus.

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. A method for preparing a liposome encapsulated nucleocapsid delivery composition, the method comprising:
(a) removing the outer envelope of an isolated non-mammalian virus to produce a purified nucleocapsid;
(b) contacting said nucleocapsid with a lipid to form a mixture, and
(c) treating said mixture to induce liposome formation,
wherein said nucleocapsid is encapsulated by said liposome and wherein said liposome encapsulated nucleocapsid is capable of delivering a nucleic acid to a mammalian cell.
